(19) United States
(12) Patent Application Publication
(10) Pub. No.: US 2025/0154179 A1
(43) Pub. Date: May 15, 2025

(54) GOLD (I) GEFITINIB DERIVATIVES AND PREPARATION METHOD AND APPLICATION THEREOF

(71) Applicants: TONGJI HOSPITAL, TONGJI MEDICAL COLLEGE, HUAZHONG UNIVERSITY OF SCIENCE AND TECHNOLOGY, Wuhan City (CN); SHANXI BETHUNE HOSPITAL (SHANXI ACADEMY OF MEDICAL SCIENCES), Taiyuan City (CN); NANJING UNIVERSITY OF CHINESE MEDICINE, Nanjing City (CN)

(72) Inventors: Shuang Wei, Wuhan City (CN); Wukun Liu, Wuhan City (CN); Lingling Wang, Wuhan City (CN); Xiaoyan Ma, Wuhan City (CN); Jia Wei, Wuhan City (CN); Xiansheng Liu, Wuhan City (CN); Xueli Bai, Wuhan City (CN); Huiguo Liu, Wuhan City (CN)

(73) Assignees: TONGJI HOSPITAL, TONGJI MEDICAL COLLEGE, HUAZHONG UNIVERSITY OF SCIENCE AND TECHNOLOGY, Wuhan City (CN); SHANXI BETHUNE HOSPITAL (SHANXI ACADEMY OF MEDICAL SCIENCES), Taiyuan City (CN); NANJING UNIVERSITY OF CHINESE MEDICINE, Nanjing City (CN)

(21) Appl. No.: 18/776,508

(22) Filed: Jul. 18, 2024

(30) Foreign Application Priority Data
Nov. 15, 2023 (CN) 202311519724.2

(51) Int. Cl. C07F 9/54; A61K 31/675

(52) U.S. Cl. CPC C07F 9/5463; A61K 31/675

(57) ABSTRACT

The invention discloses a class of gold (I) Gefitinib derivatives, their preparation methods, and applications. The methods for preparing the gold (I) Gefitinib derivatives comprise the following steps: 1) Dissolve compound I and potassium hydroxide in methanol, and add a gold ligand II to obtain a mixture; 2) Stir the mixture for reaction, and then filter under reduced pressure to obtain a crude product; 3) Purify the crude product to obtain the gold (I) Gefitinib derivatives. One of the gold (I) Gefitinib derivatives has an EGFR/TrxR dual-target anti-lung cancer effect, retaining the stem nucleus structure of Gefitinib, 4-aniline-quinazoline, to ensure its original EGFR targeting inhibition activity, and introducing alkynyl groups with different carbon chain lengths to connect different gold ligands so that it can target both EGFR and TrxR simultaneously, enhancing its anti-lung cancer activity and overcoming the resistance of lung cancer to Gefitinib.

### TECHNICAL FIELD

The present invention relates to the technical field of metal complexes and particularly to a class of gold (I) Gefitinib derivatives, their preparation methods, and applications.

### BACKGROUND ART

At present, the clinical treatment of lung cancer mainly relies on surgery combined with radiotherapy, chemotherapy and gene targeted therapy. The conventional anatomic lobectomy and mediastinal lymph node dissection are not suitable for all patients with early lung cancer. To choose radiotherapy and chemotherapy, patients' tolerance should be considered first. Patients with poor health are not tolerant. The targeted therapy is only suitable for patients with “target” lung cancer and not applicable to all patients with lung cancer. Epidermal growth factor receptor tyrosine kinase inhibitors (EGFR-TKIs) are common targeted drugs, but they are prone to drug resistance. The solution to drug resistance typically is to develop a new generation of drug according to the drug resistance mechanism of the previous generation of EGFR-TKIs. Therefore, it is necessary to conduct more intensive and extensive research on the tumor mechanism of EGFR-TKIs resistance, so as to break the cycle of drug R&D-acquired resistance-drug R&D.

Currently, introducing metal ions or metal ion binding components into biological systems to treat diseases is one of the major segments in the field of bioinorganic chemistry. A number of studies have proved that metallic drugs can be used in the treatment of tumors and even drug-resistant tumors (such as cisplatin and auranofin). Therefore, it is necessary to develop a class of dual-target EGFR-TrxR gold (I) Gefitinib derivatives to create drugs for treating lung cancer resistant to Gefitinib.

### SUMMARY OF THE INVENTION

The objective of the present invention is to address the shortcomings of the existing background art and to provide a class of gold (I) Gefitinib derivatives, their preparation methods, and applications.

In order to achieve the foregoing objective, the present invention provides a class of gold (I) Gefitinib derivatives, with the structural formula as follows:

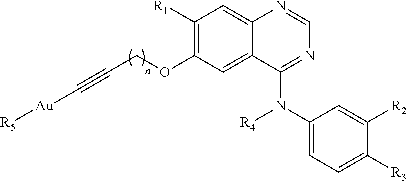

where, n is selected from 1, 2, 3, 4 and 5;
R₁ is selected from methoxy, acetyl, hydroxyl, 4-propyl morpholine and 2-methoxyethoxy;
R₂ and R₃ are selected from hydrogen, halogen and —CN;
R₄ is selected from hydrogen, alkyl and 4-propyl morpholine;
R₅ is selected from triphenylphosphine, triethylphosphine, tricyclohexylphosphine, 1,3,5-triaza-7-phosphaadamantane, 1,3-diethylimidazole, 1,3-diethyl-4,5-bis(4-methoxyphenyl)imidazole, 1,3-diethyl-4,5-bis(4-fluorophenyl)imidazole, 1-(anthracene-9-methyl)-3-ethyl-4,5-bis(4-fluorophenyl)imidazole, 1-(anthracene-9-methyl)-3-ethyl-4,5-bis(4-methoxy phenyl)imidazole, 1,3-diethyl-4,5-bis(biphenyl)imidazole and 1,3-diethyl-4,5-bis(4′-fluoro-[1,1′-biphenyl])imidazole.

The present invention further provides a method for preparing the foregoing gold (I) Gefitinib derivatives, comprising the following steps:
1) dissolving a compound I and potassium hydroxide in methanol and adding a gold ligand II to obtain a mixture;
2) stirring the mixture for reaction and then filtering under reduced pressure to obtain a crude product; and
3) purifying the crude product to obtain the gold (I) Gefitinib derivatives;
wherein the structural formula of the compound I is as follows:

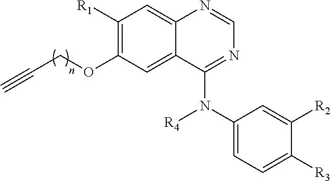

where n is selected from 1, 2, 3, 4 and 5;
R₁ is selected from methoxy, acetyl, hydroxyl, 4-propyl morpholine and 2-methoxyethoxy;
R₂ and R₃ are selected from hydrogen, halogen and —CN;
R₄ is selected from hydrogen, alkyl and 4-propyl morpholine;
The structural formula of the gold ligand II is as follows:

R₅—Au—X
where R₅ is selected from triphenylphosphine, triethylphosphine, tricyclohexylphosphine, 1,3,5-triaza-7-phosphaadamantane, 1,3-diethylimidazole, 1,3-diethyl-4,5-bis(4-methoxyphenyl)imidazole, 1,3-diethyl-4,5-bis(4-fluorophenyl)imidazole, 1-(anthracene-9-methyl)-3-ethyl-4,5-bis(4-fluorophenyl)imidazole, 1-(anthracene-9-methyl)-3-ethyl-4,5-bis(4-methoxy phenyl)imidazole, 1,3-diethyl-4,5-bis(biphenyl)imidazole and 1,3-diethyl-4,5-bis(4′-fluoro-[1,1′-biphenyl])imidazole;
X is selected from chlorine and bromine.

Further, in step 1), the solid-liquid ratio of the compound I to methanol is (0.01˜0.1) mmol:1 ml.

Preferably, in step 1), the solid-liquid ratio of the compound I to methanol is 0.0923 mmol:1 ml.

Further, in step 1), the molar ratio of potassium hydroxide to the gold ligand II is (0.2˜1):(0.02˜0.1).

Further, in step 1), the molar ratio of the compound I to the gold ligand II is (0.05˜0.1):(0.03˜0.1).

Further, in step 2), the mixture is stirred for reaction at 25˜35° C.

Preferably, in step 2), the mixture is stirred for reaction at 25° C.

Further, in step 2), the mixture is stirred for reaction for 0.5˜10 h.

Further, in step 3), the crude product is purified by means of methanol washing or silica gel column chromatography.

The present invention further provides an application of one of the foregoing gold (I) Gefitinib derivatives. The gold (I) Gefitinib derivative is used to prepare a drug treating lung cancer resistant to Gefitinib.

Compared with the prior art, the present invention has the following advantages:

The first, the gold (I) Gefitinib derivatives provided by the present invention retain the stem nucleus structure of Gefitinib, 4-aniline-quinazoline, to ensure its original EGFR targeting inhibition activity, and introduce alkynyl groups with different carbon chain lengths to connect different gold ligands (R₅—Au—X). The structure is novel.

The second, the gold (I) Gefitinib derivative provided by the present invention uses alkynyl groups to connect gold (I) and inhibitor ligand C—Au—P to make the structure more stable.

The third, the gold (I) Gefitinib derivative provided by the present invention is synthesized through reaction at room temperature for a short time. The reaction conditions are moderate and the process is simple and easy to operate.

The fourth, the stem nucleus structure of Gefitinib retained by the gold (I) Gefitinib derivative provided by the present invention makes it still have a good inhibitory effect on Gefitinib-sensitive lung cancer.

The fifth, one of the gold (I) Gefitinib derivatives provided by the present invention introduces a gold ligand, which makes it target at TrxR and GPX4 at the same time, significantly inducing the elevation of the oxidative stress level of drug resistant tumors, and showing a good effect against drug-resistant tumors.

The results of a mouse in vivo experiment further prove that one of the gold (I) Gefitinib derivatives has a certain biosafety and no obvious impact on the liver and kidney function of mice while having an effect against drug-resistant tumors.

In conclusion, the gold (I) Gefitinib derivative provided by the present invention has good stability and biosafety, the retained Gefitinib stem nucleus structure plays a role in inhibiting sensitive lung cancer, and the introduced gold ligand also has a significant inhibitory effect on drug-resistant lung cancer and shows a good prospect in treating drug-resistant lung cancer.

### BRIEF DESCRIPTION OF THE DRAWINGS

FIG. 1 is an X-ray crystal structure diagram of a product in Embodiment 8;

FIG. 2 shows the effect of the product in Embodiment 8 on inhibiting proliferation and promoting apoptosis of drug-resistant tumor cells, where FIG. A is a time curve for growth inhibition of lung cancer cells; FIG. B is a statistical diagram for inhibition of the colony formation ability of lung cancer cells; FIG. C is a statistical diagram for inhibition of the migration ability of lung cancer cells; FIG. D is a statistical diagram for an effect on promoting apoptosis of lung cancer cells;

FIG. 3 shows an effect of the product in Embodiment 8 on intervening in and inhibiting the growth of subcutaneous drug-resistant lung cancer, where FIG. A shows a subcutaneous tumor model in nude mice and a schematic diagram of administration; FIG. B is time curve for subcutaneous tumor growth of nude mice in each group; FIG. C is a histogram of subcutaneous tumors in nude mice in each group; FIG. D shows the statistical results of subcutaneous tumor tissue weight of nude mice in each group;

FIG. 4 shows the mechanism of the product in Embodiment 8 in inhibiting drug-resistant lung cancer.

### DETAILED DESCRIPTION

Below the implementation of the present invention is elaborated in conjunction with embodiments. The embodiments are not intended to limit the present invention and are examples only. Through the elaboration, the advantages of the present invention will become more apparent and understandable.

### Embodiment 1

A gold (I) Gefitinib derivative in this embodiment, with a structural formula as follows:

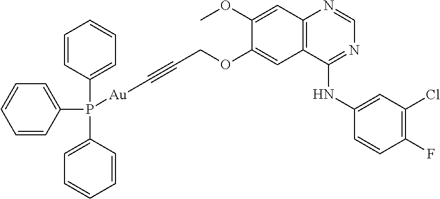

A method for preparing the gold (I) Gefitinib derivative in this embodiment, comprising the following steps:
1) dissolving a compound I (0.139 mmol) in methanol (15 ml), then adding potassium hydroxide (0.749 mmol), stirring them at room temperature for 5 min and then adding chloro (triphenylphosphine) gold (I) (0.110 mmol) to obtain a mixture;
wherein the structural formula of the compound I is as follows:

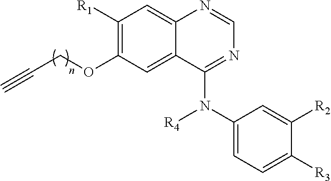

where n=1, R₁═—OCH₃, R₂═Cl, R₃═F, R₄═H;
2) stirring the mixture for reaction at 25° C. for 6 h and then filtering under reduced pressure to obtain a crude product;
3) washing the crude product with methanol to obtain the gold (I) Gefitinib derivatives, which form a white solid, with a yield of 56.58%. 1H NMR (500 MHz, DMSO-d₆) δ 9.56 (s, 1H), 8.51 (s, 1H), 8.14-8.09 (m, 1H), 7.87 (s, 1H), 7.79 (dt, J=7.7, 3.3 Hz, 1H), 7.67-7.39 (m, 16 h), 7.22 (s, 1H), 4.97 (s, 2H), 3.95 (s, 3H). 13C NMR (126 MHz, DMSO-d₆) δ 156.53, 155.12, 153.22, 147.66, 147.57, 134.38, 134.27, 132.44, 130.13, 130.04, 129.80, 123.91, 122.77, 122.71, 117.10, 116.93, 107.80, 103.72, 58.24, 56.33. ESI-MS m/z [M]⁺ calc.: 816.02 found: 816.63.

### Embodiment 2

A gold (I) Gefitinib derivative in this embodiment, with a structural formula as follows:

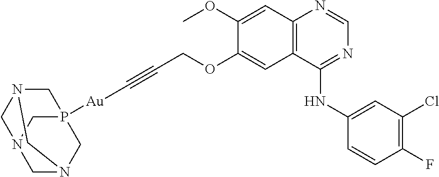

A method for preparing the gold (I) Gefitinib derivative in this embodiment, comprising the following steps:
1) dissolving a compound I (0.057 mmol) in methanol (10 ml), then adding potassium hydroxide (0.210 mmol), stirring them at room temperature for 5 min and then adding (1,3,5-triaza-7-phosphaadamantane)-gold chloride (0.033 mmol) to obtain a mixture;
wherein the structural formula of the compound I is as follows:

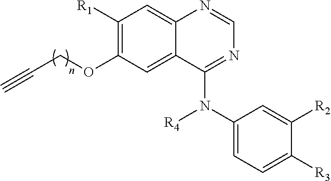

where n=1, R₁═—OCH₃, R₂═Cl, R₃═F, R₄═H;
2) stirring the mixture for reaction at 25° C. for 1.5 h and then filtering under reduced pressure to obtain a crude product;
3) washing the crude product with methanol to obtain the gold (I) Gefitinib derivatives, which form a light-yellow solid, with a yield of 66.11%. 1H NMR (500 MHZ, DMSO-d₆) δ 9.52 (d, J=2.2 Hz, 1H), 8.51 (s, 1H), 8.12 (ddd, J=7.2, 4.6, 2.6 Hz, 1H), 7.84-7.76 (m, 2H), 7.50-7.43 (m, 1H), 7.21 (s, 1H), 4.90 (s, 2H), 4.46 (d, J=12.8 Hz, 3H), 4.32 (d, J=13.0 Hz, 3H), 4.22 (s, 6 h), 3.94 (d, J=1.9 Hz, 3H). 13C NMR (126 MHZ, DMSO-d₆) δ 156.48, 155.15, 153.18, 152.63, 147.57, 147.53, 137.27, 123.78, 122.66, 122.60, 119.21, 117.14, 116.97, 109.09, 107.76, 103.78, 72.30, 72.24, 58.21, 56.32, 56.24, 51.21, 51.05. ESI-MS m/z [M+H]⁺ calc.: 711.88 found: 711.70.

### Embodiment 3

A gold (I) Gefitinib derivative in this embodiment, with a structural formula as follows:

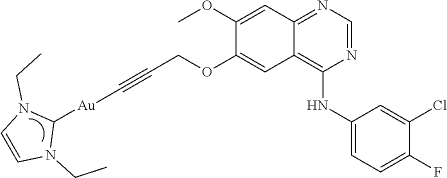

A method for preparing the gold (I) Gefitinib derivative in this embodiment, comprising the following steps:
1) dissolving a compound I (0.056 mmol) in methanol (10 ml), then adding potassium hydroxide (0.203 mmol), stirring them at room temperature for 5 min and then adding 1,3-diethylimidazole gold bromide (0.028 mmol) to obtain a mixture;
wherein the structural formula of the compound I is as follows:

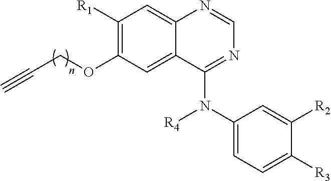

where n=1, R₁═—OCH₃, R₂═Cl, R₃═F, R₄═H;
2) stirring the mixture for reaction at 25° C. for 4 h and then evaporating the solvent to obtain a crude product;
3) subjecting the crude product to silica gel column chromatography (the eluent is EA:PE=1:10 till EA gradient elution) to obtain the gold (I) Gefitinib derivatives, which form a yellow solid, with a yield of 27.98%. 1H NMR (500 MHz, DMSO-d₆) δ 9.56 (s, 1H), 8.51 (s, 1H), 8.12 (dd, J=6.9, 2.7 Hz, 1H), 7.87-7.78 (m, 2H), 7.45 (d, J=7.8 Hz, 3H), 7.22 (s, 1H), 4.93 (s, 2H), 4.09 (q, J=7.2 Hz, 4H), 3.96 (s, 3H), 1.34 (t, J=7.3 Hz, 6 h). 13C NMR (126 MHZ, DMSO-d₆) δ 184.17, 156.51, 155.14, 153.14, 152.63, 147.82, 147.50, 123.87, 122.75, 122.16, 121.47, 117.10, 116.93, 109.15, 107.74, 103.58, 97.59, 58.66, 56.30, 46.10, 45.80, 29.50, 17.33, 17.13. ESI-MS m/z [M+H]⁺ calc.: 678.91 found: 678.53.

### Embodiment 4

A gold (I) Gefitinib derivative in this embodiment, with a structural formula as follows:

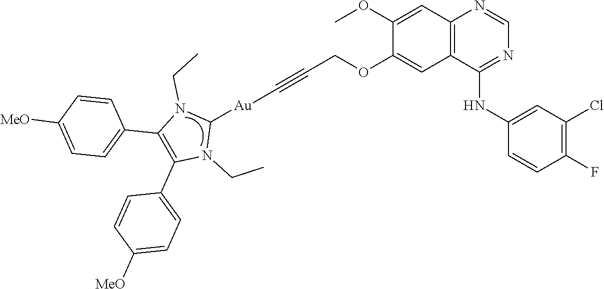

A method for preparing the gold (I) Gefitinib derivative in this embodiment, comprising the following steps:
1) dissolving a compound I (0.064 mmol) in methanol (10 ml), then adding potassium hydroxide (0.237 mmol), stirring them at room temperature for 5 min and then adding 1,3-diethyl-4,5-bis(4-methoxyphenyl)imidazole gold bromide (0.033 mmol) to obtain a mixture;
wherein the structural formula of the compound I is as follows:

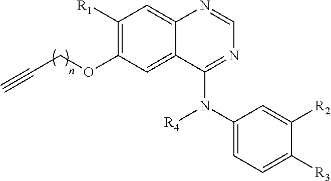

where n=1, R₁═—OCH₃, R₂═Cl, R₃═F, R₄═H;
2) stirring the mixture for reaction at 25° C. for 1 h and then filtering under reduced pressure to obtain a crude product;
3) washing the crude product with methanol to obtain the gold (I) Gefitinib derivatives, which form a white solid, with a yield of 84.95%, 1H NMR (500 MHz, DMSO-d₆) δ 9.56 (s, 1H), 8.51 (s, 1H), 8.12 (dd, J=6.8, 2.7 Hz, 1H), 7.87 (s, 1H), 7.81 (ddd, J=9.1, 4.3, 2.7 Hz, 1H), 7.45 (t, J=9.1 Hz, 1H), 7.29-7.24 (m, 4H), 7.22 (s, 1H), 6.97-6.91 (m, 4H), 4.96 (s, 2H), 4.02 (q, J=7.2 Hz, 4H), 3.96 (s, 3H), 3.75 (s, 6 h), 1.17 (t, J=7.2 Hz, 6 h). 13C NMR (126 MHZ, DMSO-d₆) δ 183.79, 160.11, 156.49, 155.13, 153.14, 147.84, 147.51, 132.50, 131.01, 123.85, 122.72, 122.67, 120.07, 117.10, 116.92, 114.61, 109.15, 107.75, 103.57, 97.76, 58.71, 56.31, 55.59, 43.90, 17.19. ESI-MS m/z [M]⁺ calc.: 890.16 found: 890.87.

### Embodiment 5

A gold (I) Gefitinib derivative in this embodiment, with a structural formula as follows:

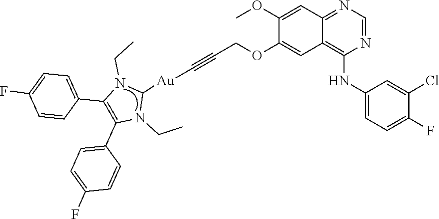

A method for preparing the gold (I) Gefitinib derivative in this embodiment, comprising the following steps:
1) dissolving a compound I (0.056 mmol) in methanol (10 ml), then adding potassium hydroxide (0.203 mmol), stirring them at room temperature for 5 min and then adding 1,3-diethyl-4,5-bis(4-fluorophenyl)imidazole gold bromide (0.028 mmol) to obtain a mixture;
wherein the structural formula of the compound I is as follows:

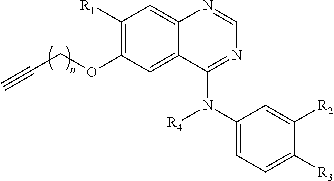

where n=1, R₁═—OCH₃, R₂═Cl, R₃═F, R₄═H;
2) stirring the mixture for reaction at 25° C. for 5 h and then evaporating the solvent to obtain a crude product
3) subjecting the crude product to silica gel column chromatography (the eluent is EA:PE=1:20 till EA:PE=1:1 gradient elution) to obtain the gold (I) Gefitinib derivatives, which form a while solid, with a yield of 39.68%. 1H NMR (500 MHZ, Chloroform-d) δ 8.51 (s, 1H), 8.47 (s, 1H), 8.42 (s, 1H), 7.67 (dd, J=6.7, 2.7 Hz, 1H), 7.48 (ddd, J=9.0, 4.2, 2.7 Hz, 1H), 6.98 (d, J=7.2 Hz, 8H), 6.89-6.80 (m, 2H), 5.73 (s, 2H), 3.90 (s, 3H), 3.59 (q, J=7.2 Hz, 4H), 0.80 (t, J=7.2 Hz, 6 h). 13C NMR (126 MHZ, Chloroform-d) δ 184.14, 164.05, 162.05, 155.40, 154.48, 152.77, 147.22, 132.24, 132.17, 130.28, 128.41, 123.29, 123.27, 122.13, 120.08, 116.23, 116.06, 115.22, 108.98, 106.93, 103.13, 103.01, 58.01, 55.92, 43.67, 16.63. ESI-MS m/z [M]⁺ calc.: 866.09 found: 866.65.

### Embodiment 6

A gold (I) Gefitinib derivative in this embodiment, with a structural formula as follows:

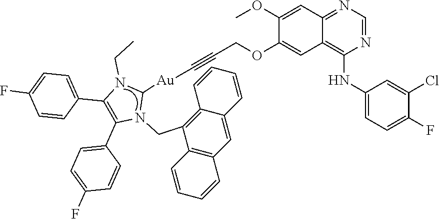

A method for preparing the gold (I) Gefitinib derivative in this embodiment, comprising the following steps:
1) dissolving a compound I (0.056 mmol) in methanol (10 ml), then adding potassium hydroxide (0.203 mmol), stirring them at room temperature for 5 min and then adding 1-(anthracene-9-methyl)-3-ethyl-4,5-bis(4-methoxy phenyl) imidazole gold chloride (0.028 mmol) to obtain a mixture;
wherein the structural formula of the compound I is as follows:

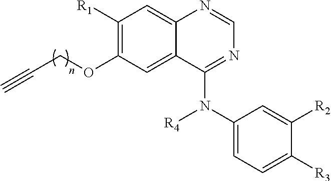

where n=1, R₁═—OCH₃, R₂═Cl, R₃═F, R₄═H;
2) stirring the mixture for reaction at 25° C. for 4.5 h and then filtering under reduced pressure to obtain a crude product;
3) washing the crude product with methanol to obtain the gold (I) Gefitinib derivatives, which form a yellow solid, with a yield of 36.73%. 1H NMR (500 MHz, DMSO-d₆) δ 9.52 (s, 1H), 8.46 (s, 1H), 8.42 (s, 1H), 8.19-8.14 (m, 2H), 8.05 (dd, J=6.8, 2.7 Hz, 1H), 7.95-7.90 (m, 2H), 7.85 (s, 1H), 7.75 (ddd, J=9.1, 4.3, 2.7 Hz, 1H), 7.41-7.33 (m, 5H), 7.21-7.15 (m, 3H), 7.10 (t, J=8.8 Hz, 2H), 6.53-6.47 (m, 2H), 6.44-6.37 (m, 4H), 4.95 (s, 2H), 4.07 (q, J=7.2 Hz, 2H), 3.92 (s, 3H), 1.15 (t, J=7.2 Hz, 3H). 13C NMR (126 MHz, DMSO-d₆) δ 186.08, 161.77, 161.01, 156.46, 155.12, 154.50, 153.04, 147.75, 147.36, 137.15, 133.25, 133.18, 132.69, 132.63, 131.44, 131.11, 130.88, 130.59, 129.37, 129.31, 126.80, 125.91, 125.20, 124.06, 123.86, 123.71, 123.48, 122.59, 122.54, 119.29, 119.15, 116.98, 116.81, 116.30, 116.13, 114.99, 114.82, 109.13, 107.55, 103.53, 97.72, 58.51, 56.26, 47.73, 44.33, 31.72, 31.59, 29.43, 29.13, 22.53, 17.14, 14.39. ESI-MS m/z [M]⁺ calc.: 1028.28 found: 1028.71.

### Embodiment 7

A gold (I) Gefitinib derivative in this embodiment, with a structural formula as follows:

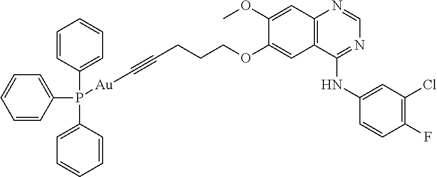

A method for preparing the gold (I) Gefitinib derivative in this embodiment, comprising the following steps:
1) dissolving a compound I (0.052 mmol) in methanol (5 ml), then adding potassium hydroxide (0.203 mmol), stirring them at room temperature for 5 min and then adding triphenylphosphine gold chloride (0.028 mmol) to obtain a mixture;
wherein the structural formula of the compound I is as follows:

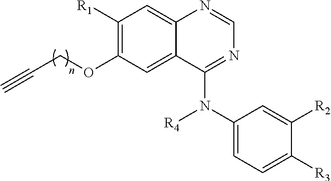

where n=3, R₁═—OCH₃, R₂═Cl, R₃═F, R₄═H.
2) stirring the mixture for reaction at 25° C. for 4.5 h and then filtering under reduced pressure to obtain a crude product;
3) washing the crude product with methanol to obtain the gold (I) Gefitinib derivatives, which form a while solid, with a yield of 69.47%. 1H NMR (500 MHz, DMSO-d₆) δ 9.58 (s, 1H), 8.50 (s, 1H), 8.09 (dd, J=6.9, 2.7 Hz, 1H), 7.84 (s, 1H), 7.78 (ddd, J=9.0, 4.3, 2.6 Hz, 1H), 7.62-7.55 (m, 9H), 7.53-7.41 (m, 7H), 7.22 (s, 1H), 4.26 (t, J=6.1 Hz, 2H), 3.95 (s, 3H), 2.42 (t, J=6.8 Hz, 2H), 1.98 (p, J=6.4 Hz, 2H). 13C NMR (126 MHz, DMSO-d₆) δ 156.49, 154.94, 153.09, 148.84, 147.44, 134.35, 134.24, 132.33, 132.31, 130.15, 130.07, 129.98, 129.71, 124.03, 122.90, 122.85, 119.29, 117.04, 116.87, 109.27, 107.77, 102.88, 67.99, 56.36, 29.29, 16.71. ESI-MS m/z [M]⁺ calc.: 844.07 found: 844.73.

### Embodiment 8

A gold (I) Gefitinib derivative in this embodiment, with a structural formula as follows:

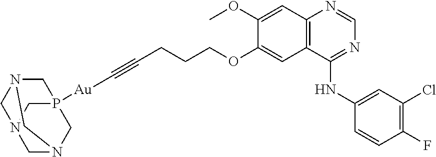

A method for preparing the gold (I) Gefitinib derivative in this embodiment, comprising the following steps:
1) dissolving a compound I (0.063 mmol) in methanol (5 ml), then adding potassium hydroxide (0.310 mmol), stirring them at room temperature for 5 min and then adding (1,3,5-triaza-7-phosphaadamantane)-gold chloride (0.050 mmol) to obtain a mixture;
wherein the structural formula of the compound I is as follows:

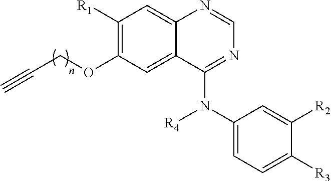

where n=3, R₁═—OCH₃, R₂═Cl, R₃═F, R₄═H.
2) stirring the mixture for reaction at 25° C. for 1 h and then filtering under reduced pressure to obtain a crude product;
3) washing the crude product with methanol to obtain the gold (I) Gefitinib derivatives, which form a while solid, with a yield of 86.42%. 1H NMR (500 MHz, DMSO-d₆) δ 9.59 (s, 1H), 8.50 (s, 1H), 8.12 (dd, J=6.8, 2.6 Hz, 1H), 7.83-7.79 (m, 2H), 7.46 (t, J=9.1 Hz, 1H), 7.21 (s, 1H), 4.47 (d, J=12.7 Hz, 3H), 4.32 (d, J=13.0 Hz, 3H), 4.21 (d, J=7.6 Hz, 8H), 3.94 (s, 3H), 2.33 (t, J=6.8 Hz, 2H), 1.92 (p, J=6.5 Hz, 2H). 13C NMR (126 MHZ, DMSO-d₆) δ 156.48, 154.94, 153.09, 148.87, 147.44, 137.28, 124.02, 122.89, 122.83, 117.08, 116.91, 109.24, 107.77, 102.80, 72.32, 72.26, 67.96, 56.35, 51.24, 51.08, 29.37, 16.72. ESI-MS m/z [M+H]⁺ calc.: 739.94 found: 739.81.

### Embodiment 9

A gold (I) Gefitinib derivative in this embodiment, with a structural formula as follows:

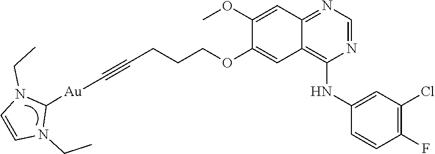

A method for preparing the gold (I) Gefitinib derivative in this embodiment, comprising the following steps:
1) dissolving a compound I (0.052 mmol) in methanol (5 ml), then adding potassium hydroxide (0.260 mmol), stirring them at room temperature for 5 min and then adding 1,3-diethylimidazole gold bromide (0.056 mmol) to obtain a mixture;
wherein the structural formula of the compound I is as follows:

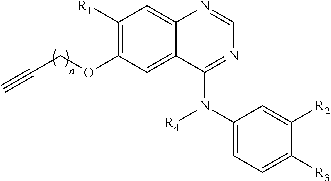

where n=3, R₁═—OCH₃, R₂═Cl, R₃═F, R₄═H.
2) stirring the mixture for reaction at 25° C. for 9 h and then filtering under reduced pressure to obtain a crude product;
3) washing the crude product with methanol to obtain the gold (I) Gefitinib derivatives, which form a yellow solid, with a yield of 15.30%. 1H NMR (500 MHz, DMSO-d₆) δ 9.60 (s, 1H), 8.50 (s, 1H), 8.11 (dd, J=6.9, 2.7 Hz, 1H), 7.89-7.75 (m, 2H), 7.43 (d, J=16.2 Hz, 3H), 7.21 (s, 1H), 4.26 (t, J=6.0 Hz, 2H), 4.08 (q, J=7.3 Hz, 5H, OCH₃), 3.95 (s, 2H), 2.39 (d, J=6.7 Hz, 2H), 1.95 (t, J=6.5 Hz, 2H), 1.33 (t, J=7.2 Hz, 6 h). 13C NMR (126 MHZ, DMSO-d₆) δ 185.03, 172.64, 156.48, 154.95, 153.04, 148.91, 147.42, 137.29, 123.99, 122.86, 122.81, 122.15, 121.30, 117.04, 116.87, 107.75, 102.81, 68.12, 60.23, 56.34, 46.10, 45.72, 29.59, 21.63, 17.33, 17.09. ESI-MS m/z [M+H]⁺ calc.: 706.97 found: 706.58.

### Embodiment 10

A gold (I) Gefitinib derivative in this embodiment, with a structural formula as follows:

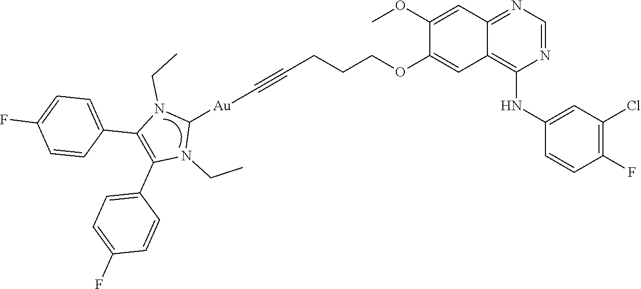

A method for preparing the gold (I) Gefitinib derivative in this embodiment, comprising the following steps:
1) dissolving a compound I (0.028 mmol) in methanol (5 ml), then adding potassium hydroxide (0.135 mmol), stirring them at room temperature for 5 min and then adding 1,3-diethyl-4,5-bis(4-methoxyphenyl)imidazole gold bromide (0.020 mmol) to obtain a mixture;
wherein the structural formula of the compound I is as follows:

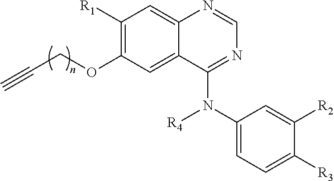

where n=3, R₁═—OCH₃, R₂═Cl, R₃═F, R₄═H.
2) stirring the mixture for reaction at 25° C. for 5 h and then filtering under reduced pressure to obtain a crude product;
3) washing the crude product with methanol to obtain the gold (I) Gefitinib derivatives, which form a while solid, with a yield of 83.16%. 1H NMR (500 MHz, DMSO-d₆) δ 9.59 (s, 1H), 8.50 (s, 1H), 8.12 (dd, J=6.9, 2.7 Hz, 1H), 7.86 (s, 1H), 7.82 (ddt, J=9.1, 4.3, 2.0 Hz, 1H), 7.44 (t, J=9.1 Hz, 1H), 7.30-7.20 (m, 5H), 6.97-6.91 (m, 4H), 4.27 (t, J=6.1 Hz, 2H), 4.02 (q, J=7.2 Hz, 4H), 3.96 (s, 3H), 3.75 (s, 6 h), 2.41 (t, J=6.9 Hz, 2H), 1.97 (p, J=6.6 Hz, 2H), 1.15 (t, J=7.2 Hz, 6 h). 13C NMR (126 MHZ, DMSO-d₆) δ 184.68, 160.08, 156.48, 154.97, 153.05, 147.42, 137.28, 132.48, 130.85, 123.98, 122.86, 122.80, 121.29, 120.16, 119.14, 117.05, 116.88, 114.60, 109.26, 107.76, 103.04, 102.82, 68.15, 56.36, 55.58, 43.82, 29.55, 17.14, 17.10. ESI-MS m/z [M]⁺ calc.: 918.22 found: 918.87.

### Embodiment 11

A gold (I) Gefitinib derivative in this embodiment, with a structural formula as follows:

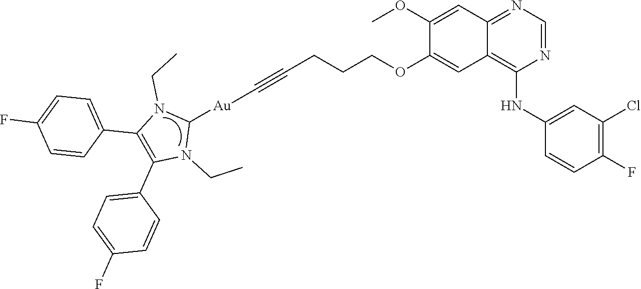

A method for preparing the gold (I) Gefitinib derivative in this embodiment, comprising the following steps:
1) dissolving a compound I (0.041 mmol) in methanol (5 ml), then adding potassium hydroxide (0.232 mmol), stirring them at room temperature for 5 min and then adding 1,3-diethyl-4,5-bis(4-fluorophenyl)imidazole gold bromide (0.031 mmol) to obtain a mixture;
wherein the structural formula of the compound I is as follows:

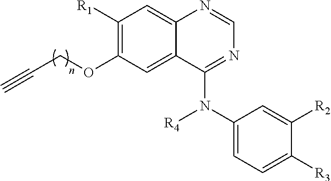

where n=3, R₁═—OCH₃, R₂═Cl, R₃═F, R₄═H;
2) stirring the mixture for reaction at 25° C. for 4 h and then filtering under reduced pressure to obtain a crude product;
3) washing the crude product with methanol to obtain the gold (I) Gefitinib derivatives, which form a while solid, with a yield of 54.84%. 1H NMR (500 MHz, DMSO-d₆) δ 9.59 (s, 1H), 8.50 (s, 1H), 8.12 (dd, J=6.9, 2.6 Hz, 1H), 7.86 (s, 1H), 7.82 (ddd, J=9.0, 4.3, 2.6 Hz, 1H), 7.47-7.38 (m, 5H), 7.29-7.20 (m, 5H), 4.27 (t, J=6.2 Hz, 2H), 4.04 (q, J=7.2 Hz, 4H), 3.96 (s, 3H), 2.41 (t, J=6.8 Hz, 2H), 1.97 (p, J=6.4 Hz, 2H), 1.16 (t, J=7.2 Hz, 6 h). 13C NMR (126 MHz, DMSO-d₆) δ 185.33, 161.92, 156.48, 154.97, 153.05, 148.91, 147.43, 137.31, 133.54, 133.48, 130.45, 123.97, 122.85, 117.05, 116.88, 116.42, 116.25, 109.26, 107.78, 102.83, 68.15, 56.36, 44.00, 29.56, 17.06. ESI-MS m/z [M]⁺ calc.: 894.15 found: 894.70.

### Effect Example

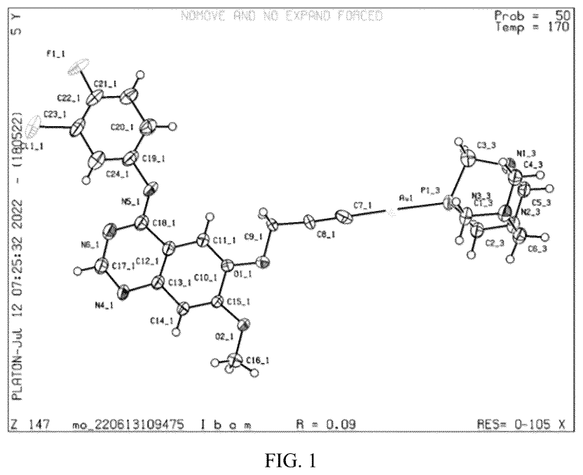

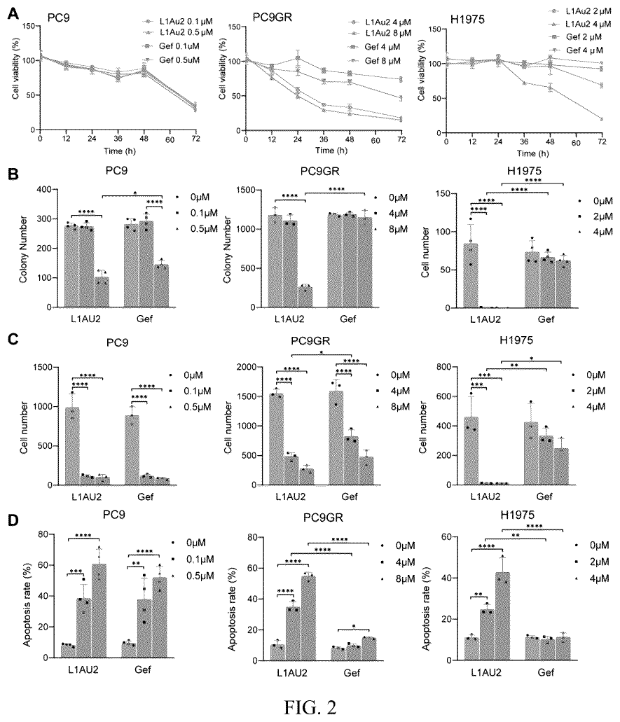

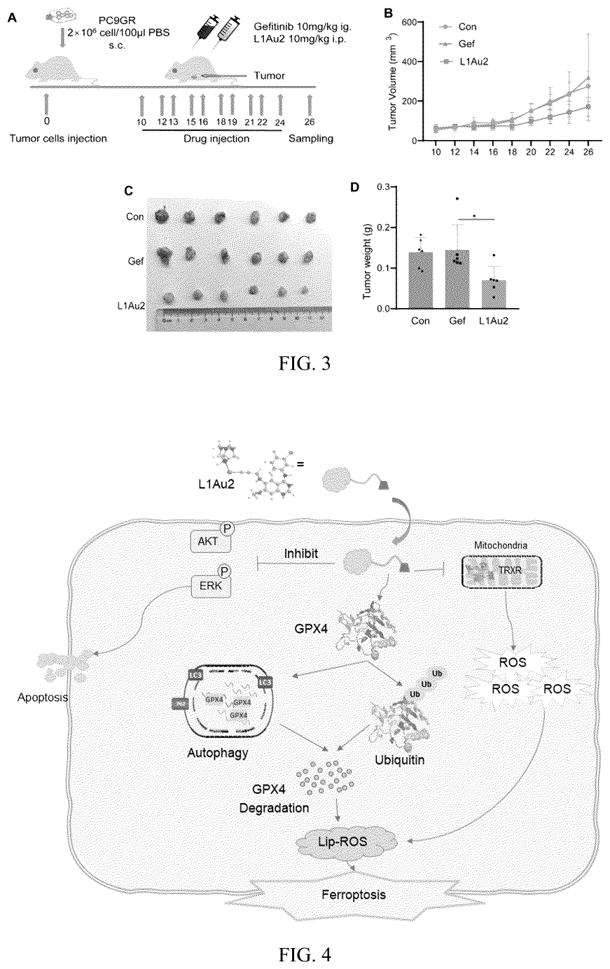

The present invention further provides an application of a gold (I) Gefitinib derivative, mainly providing the study of the effect of Embodiment 8 (L1Au2 for short). FIG. 1 shows the specific structure of L1Au2 as presented by X-ray crystal analysis. L1Au2 is mainly used to treat lung cancer resistant to first- and second-generation EGFR-TKI. In vitro experiments on L1Au2 were conducted against Gefitinib-sensitive and Gefitinib-resistant lung cancer. The results show that it has an obvious effect on inhibiting proliferation and promoting apoptosis of drug-resistant lung cancer. The cell lines used in the experiment are internationally universal lung cancer tumor cell lines: Gefitinib-sensitive lung cancer cell line PC9, and Gefitinib-resistant cell lines PC9GR and H1975. CCK-8 and plate cloning experiments found that complexes had an obvious inhibitory effect on the proliferation of these three lung cancer cell lines, Annexin V-FITC/PI double staining assay found that it promoted apoptosis of lung cancer cells, and Transwell experiment determined that it inhibited lung cancer cell migration. See FIG. 2 for the effects of the product, where FIG. A: CCK8 showed that L1Au2-induces lung cancer cell death in a time-dependent manner; FIG. B: the colony stimulation experiment showed that L1Au2 significantly inhibits the colony formation ability of lung cancer cells; FIG. C: L1Au2 significantly inhibited the migration ability of lung cancer cells. Statistical results of the Transwell experiment; FIG. D: L1Au2 significantly promoted the apoptosis of lung cancer cells. The gold (I) Gefitinib derivative significantly inhibited the growth of drug-resistant subcutaneous tumors in nude mice. See FIG. 3 for the effect diagram of a specific product. L1Au2 not only inhibits EGFR-related pathway activation, inhibits thioredoxin reductase activity and induces intracellular mitochondrial damage, but also degrades GPX4 protein by promoting autophagy and inducing GPX4 protein ubiquitination and severely induces elevation of the intracellular oxidative stress level, thus resulting in ferroptosis of Gefitinib-resistant lung cancer cells, which shows an effect on multi-target inhibition of Gefitinib-resistant lung cancer. In conclusion, L1Au2 performs better than Gefitinib against drug-resistant tumors in vivo and in vitro in multiple targets, and its underlying mechanism is shown in FIG. 4.

The above are only some embodiments of the present invention. It should be noted that all the variations or replacements within the scope of the technology revealed by the present, which can be readily thought of by any person skilled in the art, shall fall within the scope of protection of the present invention, and the rest not described in detail is prior art.

1-10. (canceled)

11. A gold (I) Gefitinib derivative, wherein the gold (I) Gefitinib derivative has a structural formula as follows: 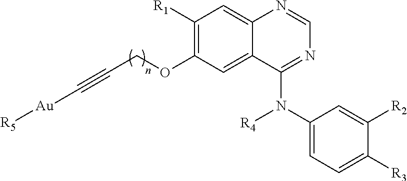
where n is selected from 1, 2, 3, 4 and 5;
R₁ is selected from methoxy, acetyl, hydroxyl, 4-propyl morpholine and 2-methoxyethoxy;
R₂ and R₃ are selected from hydrogen, halogen and —CN;
R₄ is selected from hydrogen, alkyl and 4-propyl morpholine; and
R₅ is selected from triphenylphosphine, triethylphosphine, tricyclohexylphosphine, 1,3,5-triaza-7-phosphaadamantane, 1,3-diethylimidazole, 1,3-diethyl-4,5-bis(4-methoxyphenyl)imidazole, 1,3-diethyl-4,5-bis(4-fluorophenyl)imidazole, 1-(anthracene-9-methyl)-3-ethyl-4,5-bis(4-fluorophenyl)imidazole, 1-(anthracene-9-methyl)-3-ethyl-4,5-bis(4-methoxy phenyl)imidazole, 1,3-diethyl-4,5-bis(biphenyl)imidazole and 1,3-diethyl-4,5-bis(4′-fluoro-[1,1′-biphenyl])imidazole.

12. A method for preparing the gold (I) Gefitinib derivative according to claim 11, wherein the method comprises the following steps:
1) dissolving a compound I and potassium hydroxide in methanol and adding a gold ligand II to obtain a mixture;
2) stirring the mixture for reaction and then filtering under reduced pressure to obtain a crude product;
3) purifying the crude product to obtain the gold (I) Gefitinib derivatives;
wherein the structural formula of the compound I is as follows: 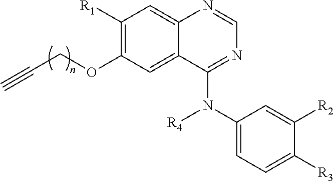
where n is selected from 1, 2, 3, 4 and 5;
R₁ is selected from methoxy, acetyl, hydroxyl, 4-propyl morpholine and 2-methoxyethoxy;
R₂ and R₃ are selected from hydrogen, halogen and —CN;
R₄ is selected from hydrogen, alkyl and 4-propyl morpholine;
wherein the structural formula of the gold ligand II is as follows:
R₅—Au—X
where R₅ is selected from triphenylphosphine, triethylphosphine, tricyclohexylphosphine, 1,3,5-triaza-7-phosphaadamantane, 1,3-diethylimidazole, 1,3-diethyl-4,5-bis(4-methoxyphenyl)imidazole, 1,3-diethyl-4,5-bis(4-fluorophenyl)imidazole, 1-(anthracene-9-methyl)-3-ethyl-4,5-bis(4-fluorophenyl)imidazole, 1-(anthracene-9-methyl)-3-ethyl-4,5-bis(4-methoxy phenyl)imidazole, 1,3-diethyl-4,5-bis(biphenyl)imidazole and 1,3-diethyl-4,5-bis(4′-fluoro-[1,1′-biphenyl])imidazole; and
X is selected from chlorine and bromine.

13. The preparation method according to claim 12, wherein in step 1), the solid-liquid ratio of the compound I to methanol is (0.01˜0.1) mmol:1 ml.

14. The preparation method according to claim 13, wherein in step 1), the solid-liquid ratio of the compound I to methanol is 0.0923 mmol:1 ml.

15. The preparation method according to claim 12, wherein in step 1), the molar ratio of potassium hydroxide to the gold ligand II is (0.2˜1):(0.02˜0.1).

16. The preparation method according to claim 12, wherein in step 1), the molar ratio of the compound I to the gold ligand II is (0.05˜0.1):(0.03˜0.1).

17. The preparation method according to claim 12, wherein in step 2), the mixture is stirred for reaction at 25˜35° C.

18. The preparation method according to claim 17, wherein in step 2), the mixture is stirred for reaction for 0.5˜10 h.

19. The preparation method according to claim 12, wherein in step 3), the crude product is purified by means of methanol washing or silica gel column chromatography.

20. An application of the gold (I) Gefitinib derivative according to claim 11, wherein the gold (I) Gefitinib derivative is used to prepare a drug treating lung cancer resistant to EGFR-TKI.
